(19) United States
(12) Patent Application Publication
(10) Pub. No.: US 2018/0168811 A1
(43) Pub. Date: Jun. 21, 2018

(54) NOVEL BIODEGRADABLE AND NON-BIODEGRADABLE 3D PRINTED IMPLANTS AS A DRUG DELIVERY SYSTEM

(71) Applicant: Rowan University, Glassboro, NJ (US)

(72) Inventors: Shivakumar Iyer RANGANATHAN, Voorhees, NJ (US); Tae Won Benjamin KIM, Voorhees, NJ (US); Daniel Joseph CAMPBELL, Flanders, NJ (US); Garrett Christopher SMITH, Palm Beach Gardens, FL (US)

(73) Assignee: Rowan University, Glassboro, NJ (US)

(21) Appl. No.: 15/736,885

(22) PCT Filed: Jun. 15, 2016
(86) PCT No.: PCT/US16/37614

§ 371 Date: Dec. 15, 2017

Related U.S. Application Data
(60) Provisional application No. 62/175,572, filed on Jun. 15, 2015.

(51) Int. Cl. A61F 2/28; A61B 17/80; A61M 31/00; A61B 5/00; A61L 31/06; A61L 31/02; A61L 31/14; A61L 31/16; A61K 31/7036; A61K 31/4178

(52) U.S. Cl. CPC A61F 2/28; A61B 17/8052; A61M 31/002; A61B 5/0064; A61L 31/06; A61L 31/022; A61F 2002/30062; A61L 31/16; A61L 31/146; A61K 31/7036; A61K 31/4178; A61F 2002/2817; A61L 31/148

(57) ABSTRACT

The present invention is directed to a medical implant, methods of use and making of such implants. A method of making an implantable device may include obtaining an anatomical model in a computer aided design (CAD) system, customizing the anatomical model per patient specific parameters and creating a virtual image of the anatomical model, incorporating at least one microchannel geometry within said anatomical model, adjusting the density infill to a measurement ranging per patient specific parameters, and using three-dimensional printing to form the implantable device based on the anatomical model. The implant can be made of suitable metallic or polymeric material, such as PLA.

### CROSS-REFERENCE TO RELATED APPLICATION

This application is a national phase of International Application No. PCT/US2016/037614, filed Jun. 15, 2016, which claims the benefit of priority of U.S. Provisional Patent Application No. 62/175,572, filed Jun. 15, 2015, the entire disclosure of both of which are hereby incorporated herein by reference.

### FIELD OF THE INVENTION

The present invention relates to medical implants, apparatuses, systems and methods of using and making such implants and apparatus by employing three-dimensional printing methodologies.

### BACKGROUND OF THE INVENTION

Three-dimensional (3D) printing is a process for printing or building parts of objects in layers to produce a three-dimensional object. Various systems have been developed for three-dimensional printing. In such systems, a predetermined configuration may be designed by the way of Computer Aided Design (CAD) system connected to the printing systems and the configuration is ultimately constructed by material used for constructing support structures for a desired model.

It is anticipated that healthcare costs are currently over 18% of the Gross Domestic Product (GDP) and more specifically, costs associated with musculoskeletal diseases account for 5.7% of the GDP. According to one estimate, the expenditures related to total knee replacement alone exceed $11 billion annually and is approaching 1% of GDP. A primary contributing factor to this cost burden is the need to have numerous implant shapes/sizes to match the patient's anatomy.

In orthopedic trauma surgery, intramedullary rods are used frequently to act as internal splints in long bones such as femur and tibia until the fracture heals. When a femoral or tibial rod is infected, treatment consists of removal of the rod, surgical debridement of the bone and placement of a temporary rod loaded with antibiotics. Another contributing factor to the high cost burden relates to post-surgical infections associated with knee and hip surgeries. Osteomyelitis caused by orthopedic surgery is typically treated with removal of the implant, surgical debridement, and placement of a material, which acts as a delivery vehicle for high dose local antibiotics to the bone.

Currently, cement blocks infused with antibiotics are placed at the infection site for six weeks, along with a general antibiotic source administered daily. The most commonly used material is PMMA or bone cement. The antibiotic of choice is loaded into the PMMA at the time the polymer and monomer are mixed, and then the material is fashioned into the necessary shape or size. However, the rate of antibiotic release for the cement blocks is not only subpar, but also uncontrollable, leading to substantial degree of clinical failure.

Similarly, in joint replacement surgery, large articulating metal implants are utilized to treat osteoarthritis. The gold standard treatment when an infection occurs is a two-stage process whereby the implant is removed and a temporary spacer made of poly methyl metacrylate (PMMA) with antibiotics is inserted. The patient receives a 6-8 week course of intravenous antibiotics, and then returns to surgery for a re-implantation of a new joint replacement. Unfortunately, even with this aggressive treatment regimen, re-infection rates are high.

In general, the use of PMMA as a drug delivery mechanism has the following problems: (a) when used in joint replacement setting, patients are left non-weight bearing on the extremity for 6-8 weeks, frequently placed in casts and have problems with cement dislodging and bone erosion; (b) drug elution properties of PMMA have been shown to be poor; (c) the implant is also susceptible to bacterial colonization and becomes a nidus for continued infection; finally (d) polymerization reaction for PMMA is highly exothermic limiting the choice of antibiotics that can be mixed into the cement. Thus, there is a need for a new and improved drug delivery material and implants. The present invention addresses such need.

### SUMMARY OF THE INVENTION

The present invention is directed to a medical implant, apparatuses, systems, methods of use and making such implants, wherein the implant is made of suitable metallic or polymeric material to achieve optimal therapeutic outcome.

In at least one aspect of the present invention, delivery systems for delivering active therapeutic agents or biological material to a surgical site are provided. In one embodiment, the described implants comprise a body corresponding to an anatomical structure having plurality of microtubules, wherein the body comprises a biocompatible material. In another embodiment, the body can contain at least one or a plurality of reservoirs to store a therapeutically active ingredient for continuous delivery to a desired site. In another embodiment, the body of such implant is of polymeric or metallic material and can be a matrix that is 100% solid or up to 95% porous.

In one embodiment, the present smart implant contains a body that is made of polymeric material comprising a melt processable polymer derived from a biodegradable, bioresorbable polymer and the polymeric body comprises a juxta-articular and/or shaft region having one or more holes for receiving bone fasteners; and a head region extending from the shaft region and having a plurality of holes for receiving bone fasteners. In at least one embodiment, the fastener is of same or different polymeric content as the body.

In yet another embodiment, the body further comprising at least one reservoir and one microchannel or a network of reservoir and microchannels for storing and delivering a therapeutically active agent. In another embodiment, the body comprises a plurality of reservoirs or micro and/or nanotubules. Such reservoir and microchannels are designed in such fashion wherein the therapeutically active drug is delivered at a rate and concentration to maximize intended therapeutic results. In at least one embodiment, the therapeutically active agent is released at a controlled rate from the solid support to the site at risk of developing a post surgical infection and maintain local minimum inhibitory concentration of a desired pathogenic agent for at least up to 12 months, preferably up to 6 months and more preferably up to at least 3 months post surgery.

In one embodiment, a method of making an implantable device may include obtaining an anatomical model in a computer aided design (CAD) system, customizing the anatomical model per patient specific parameters and creating a virtual image of the anatomical model, incorporating at least one microchannel geometry within said anatomical model, adjusting the density infill to a measurement ranging per patient specific parameters, and using three-dimensional printing to form the implantable device based on the anatomical model.

In one embodiment, the method of making the implantable device may further include incorporating at least one reservoir geometry within the anatomical model or virtual design of the implant. In one embodiment, the method of using three-dimensional printing may include developing the implantable device layer by layer.

In some embodiments, the delivery of an active therapeutic agent may be accomplished through independent layers or reservoirs within the body matrix interspersed throughout the entire structure of the implant. In one another embodiment, the therapeutically active agent are dissolved or suspended in a pharmaceutically suitable vehicle and then sprayed or coated on polymeric beads such as PLA beads. In a preferred embodiment, the suitable vehicle may include water, aqueous solution or an oil such as silicone oil, in effective amounts to avoid clumping during the coating process. In yet another embodiment, the coating process can be repeated to cover beads with another additive layer.

In another embodiment, subsequent to the bead coating process, the beads are put through the Extrusion process, to create filament for 3-D printing. In a preferred embodiment, the filaments are stored in a sterile environment and kept at low temperatures until printing so that they remained unsoiled. In another embodiment, temperature setting on the printer may range between 100-400° Celsius, preferably in the ranges of 100-350, and more preferably in ranges between 200-250° Celsius, to allow the polymer, such as the PLA to undergo the printing process.

In another embodiment, the implant is made of polymeric material such as poly lactides (PLA), polyamides, polyesters, polycaprolactone (PCL), polyglycolide-co-caprolactone, polyethylene oxide (PEO), polypropylene oxide (PPO), polyglycolide-co-trimethylene carbonate (PGA-co-TMC), poly(lactic-co-glycolic acid) (PLGA), polyglycolic acid (PGA), poly-L-lactide (PLLA), polyethylene glycol (PEG), polypropylene (PP), polyethylene (PE), polyetheretherketones (PEEK), glycosaminoglycans (GAG), poly methyl metacrylate (PMMA) and combinations thereof, but most preferably of PLA.

In yet another embodiment, the implant of the present invention may be made of metals such as titanium, stainless, steel, cobalt, chrome and any combinations thereof or modified metal surfaces such as with amine, carboxylate, azide, alkyne, thiol, or maleimide groups.

In other embodiments methods of making and using such implants are described.

In yet another embodiment, kits and systems for making such implants are described.

### BRIEF DESCRIPTION OF THE DRAWING

FIG. 1A depicts a CAD model of the fragment plate with micro-channels according to one embodiment. FIG. 1B depicts a 3D printed fragment plate using natural PLA (15%) according to one embodiment. FIG. 1C depicts a 3D printed metallic fragment plate.

FIG. 2 depicts a diagram of designing and making of implantable device according to some embodiments.

FIGS. 3A, 3B, 3C and 3D depict an anatomical model of a femur plate according to an embodiment. FIG. 3E depicts a 3D printed femur plate according to some embodiments.

FIGS. 4A and 4B depict an anatomical model of a knee replacement according to an embodiment.

FIG. 4C depicts a 3D printed knee replacement according to some embodiments.

FIG. 5 illustrates various components of a total knee replacement.

FIGS. 6A, 6B, 6C and 6D depict an anatomical model of the femoral component of a total knee replacement according to an embodiment.

FIGS. 7A, 7B, 7C and 7D depict an anatomical model of the spacer of a total knee replacement according to an embodiment.

FIGS. 8A, 8B, 8C and 8D depict an anatomical model of the tibial component of a total knee replacement according to an embodiment.

FIG. 9 depict a 3D printed total knee replacement according to some embodiments.

FIG. 10 depicts an anatomical model of a pelvic plate according to an embodiment.

FIGS. 11A-11D depict a 3D printed plastic liner according to an embodiment.

FIG. 12 depicts a graph showing the effect of percentage infill according to some embodiments.

FIGS. 13A and 13B depict various mechanical properties of components made of PLA and PMMA according to some embodiments.

FIG. 14 depicts various embodiments of one or more electronic device for implementing the various methods and processes described herein.

### DETAILED DESCRIPTION OF THE PRESENT INVENTION

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. As used in this document, the term “comprising” means “including, but not limited to.”

The terms “subject,” “individual,” and “patient,” used interchangeably herein, refers to any subject, generally a mammal (e.g., human, canine, feline, equine, bovine, rodent, etc.), in need of an implant or reconstructive surgery or at risk of post-surgical complications.

The term “implant” or “implant device” used interchangeably herein and refers to any device to be implanted between bony, cartilaginous or soft tissues or between prosthetic surfaces to restore or create a gap or to replace a missing biological structure. Examples of such include pins, rods, screws, plats, used to anchor fractured bones while they heal. Other examples include intramedullary rods, hip implants, knee implants, femoral or tibial nails, prosthesis or components thereof.

The term “therapeutically effective amount” is meant an amount of a therapeutic agent, or a rate of delivery of a therapeutic agent, effective to facilitate a desired therapeutic effect.

The term ““controlled release” as used herein is meant to encompass release of a substance (e.g., a therapeutically active ingredient, a biological product, anticoagulant, etc) at a selected or otherwise controllable rate, interval, and/or amount. As such, the delivery of a liquid or semisolid active ingredient at a volume rate of from about 0.001 μl/day to about 500 μl/day, preferably about 0.01 μl/day to about 250 μl/day, more preferably about 0.1 μl/day to about 100 μl/day.

The terms “bioresorbable” and/or “biodegradable” are used interchangeably herein to refer to a material that is dissolvable in physiological conditions by physiological enzymes and/or chemical conditions. They include such polymers as poly lactides (PLA), polyamides, polyesters, polycaprolactone (PCL), polydioxanone (PDX), and the like.

In one aspect of the invention, the inventors employ three-dimensional (3-D) printing as a new and novel method of preparing customized implants or prosthesis for treatment of various bone or anatomical abnormalities. In at least one embodiment of the present invention, 3D printing is described in designing personalized healthcare products. Broadly speaking, at least one aspect of the present invention is directed to preparing smart orthopedic implants such as intramedullary rods, fragment plates and total knee implants that will address the problem of implant infections and offer an alternative to the current standard of care which is based on bone cement or other material frequently associated with adverse effects hampering the success rate in surgical procedures. At least one failure in the art is the use of material such as polymethylmethacrylate (PMMA), hydroxyappetite that either are inflexible or prone to post-surgical infections. The present invention addresses this shortcoming.

In at least one embodiment, fused deposition modeling (FDM) techniques are employed as a method of three-dimensional printing involving heating filament and building an object layer-by layer from bottom to top. In other embodiments, other types of printing such as stereolithography (SLA) and direct metal laser sintering (DMLS) can also be used. In some embodiment, an implant or prosthesis body is a copy of the native anatomical structure. Such copy of a native anatomical structure may be an implant or a knee prosthetic joint being of a standard shape that is available in different sizes, In another embodiment such implant or prosthetic joint may be customized to fit the patient's specific anatomical needs. In one embodiment for example, an exact copy of the patient's native anatomical structure is provided using a 3D-prototyping based on tomographic imaging techniques (e.g. CT-scans) or Magnetic Resonance Imaging and then 3-D printing of the prototyped model.

Three-dimensional printing which may include, for example, a hot melt printing technique or a printing technique with intermediate curing of a printed layer with actinic radiation, such as UV-radiation. The 3D-printing technique may also be a combination of hot melt printing and intermediate curing with actinic radiation. Such techniques require printable biocompatible materials or precursors which are able to react or to be cured after being printed. Suitable materials for this purpose may comprise UV-curable groups or have a melting point of between 25° C. and 200° C. or preferably 50° C. and 150° C., (for use in hot melt printing). At least one advantage of these embodiments is that it provides more comfort to the patient because once the implant or prosthesis assembly has been implanted, and the trauma has healed, the knee joint comprising the prosthesis, closely resembles the knee joint with the original native anatomical structure.

In yet another embodiment, the polymeric body comprises a juxta-articular and/or shaft region having one or more holes for receiving bone fasteners; and a head region extending from the shaft region and having a plurality of holes for receiving bone fasteners. In yet another embodiment, the body further comprising reservoirs and microchannels for storing and delivering an active therapeutic agent. Such reservoir and microchannels network are designed in such fashion wherein the active therapeutic agent is delivered at a rate and concentration to maximize intended therapeutic results. For example if the lowest concentration of an antimicrobial that will inhibit visible growth of a microorganism is determined to be of X value, then the reservoir and microtubule infrastructure of the presently described implant maintains a concentration of at least X in the area of interest for at least 6 weeks, preferably for at least 3, 6, or 9 months, and more preferably for at least 12 months.

In another embodiment, the therapeutically active agent is released at a controlled rate from the body of the implant to the site at risk of developing a post-surgical infection to not only maintain local minimum inhibitory concentration of a desired pathogenic agent, but also control pain, inflammation or any other desired local clinical outcome for at least up to 6 weeks, preferably for at least 3, 6, or 9 months, and more preferably for at least 12 months post-surgery.

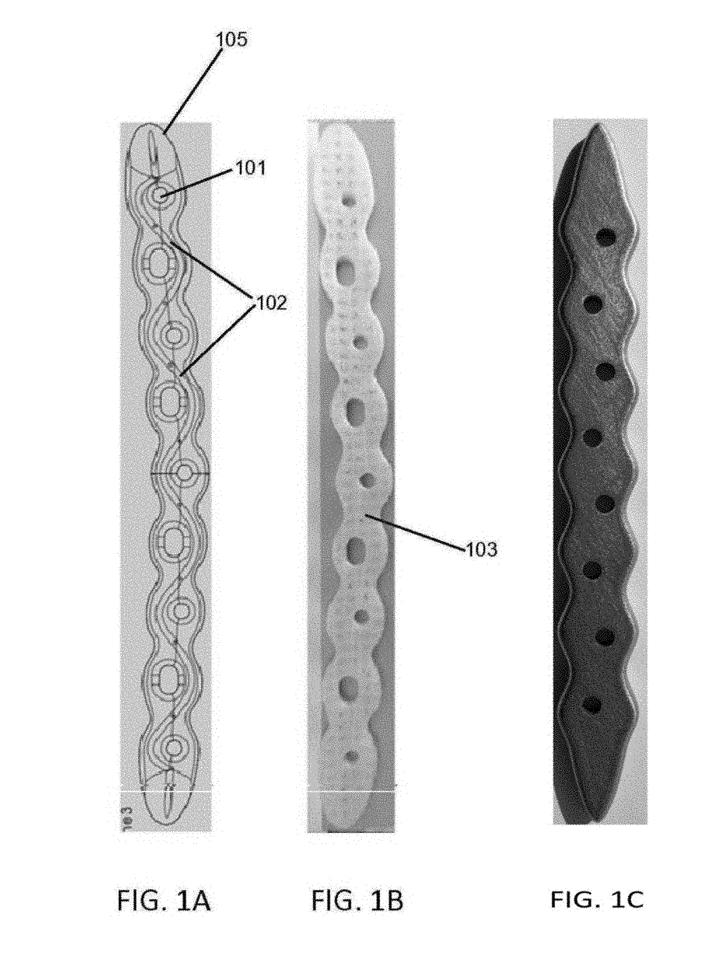

With further reference to FIG. 1A, in one embodiment, the implant of the present invention may be a femur plate and it may include holes 101 in such shapes as circular, elliptical and/or any combinations thereof to accept locking and non-locking screws. In one embodiment, the circular holes are threaded or unthreaded screw holes having a diameter ranging from 0.1-5 cm. In another embodiment, fasteners used in conjunction with the implant are of polymeric material. In a more preferred embodiment, the fasteners are of the same polymeric material as the body of the implant.

With further reference to FIG. 1B, in another aspect of the present invention, the body of the presently claimed invention can exist in a porous or solid matrix, wherein the polymeric density ranges from about 5 to 100% infill. In a more preferred embodiment, the percentage infill is 7.5, 10, 15, 20, 25, 30, 35, 40, 45, 50, 55, 60, 65, 70, 75, 80, 85 or 90% and there between. In one embodiment, the infill can have at least partial or full metallic materials. With reference to FIG. 1C, a 3D printed metallic fragment plate is shown.

In another embodiment, the polymeric material is selected from the group consisting of poly lactides (PLA), polyamides, polyesters, polycaprolactone (PCL), polydioxanone (PDX), and the like. polyglycolide-co-caprolactone, polyethylene oxide (PEO), polypropylene oxide (PPO), polyglycolide-co-trimethylene carbonate (PGA-co-TMC), poly(lactic-co-glycolic acid) (PLGA), polyglycolic acid (PGA), poly-L-lactide (PLLA), polyethylene glycol (PEG), polypropylene (PP), polyethylene (PE), polyetheretherketones (PEEK), poly(ester-ether), poly(L-leucine), poly(L-lysine), poly(amino acids), glycosaminoglycans (GAG), polymethylmethacrylate (PMMA), and combinations thereof. In a more preferred embodiment the polymer is PLA.

In yet another embodiment, the smart implant of the present invention may be made of metals such as titanium, stainless, steel, cobalt, chrome and any combinations thereof or modified metal surfaces such as with amine, carboxylate, azide, alkyne, thiol, or maleimide groups.

In another embodiment, the implant of the present invention may further comprises an active therapeutic agent, a constructive adjuvant, an osteogenic biologics, chondrogenic proteins or peptides, bone cells, osteoblasts, stem cells, demineralized bone powder; collagen, insoluble collagen derivatives or any combinations thereof. In at least one embodiment, the active therapeutic agents are selected from the group consisting of antibiotics, anabolic steroids, analgesics, antihistamines, anti-arrhythmia agents, antihypertensives, antiasthmatics, antibacterial agents, antifungal agents, anticonvulsants, anticoagulants, antihyperglycemic agents, anti-inflammatories, antineoplastics, antiparasitics, antipyretics, antispasmodics, antiviral agents, anti-uricemic agents, blood glucose-lowering agents, chemotherapeutic agents, cholesterol-reducing agents, coronary dilators, erythropoietic drugs, fungicides, growth regulators, hormone replacement agents, mineral supplements, narcotics, neuromuscular drugs, non-steroidal anti-inflammatories (NSAIDs), nutritional additives, peripheral vasodilators, therapeutic polypeptides, prostaglandins, anti-restless leg syndrome agents, steroids, uterine relaxants, vaginal preparations, vasoconstrictors, vasodilators, vitamins, local anesthetics; imaging agents, wound healing agents, and combinations thereof.

In one embodiment, the porous matrix of the implant may be made of material that contains a therapeutic agent or biological material. As such, these material may be imbibed in the porous matrix of the implant or be infused into the polymeric structure. Examples of such therapeutic agents or biological material include but are not limited to antimicrobials and/or antibiotics such as erythromycin, bacitracin, neomycin, penicillin, polymycin B, tetracyclines, biomycin, chloromycetin, and streptomycins, cefazolin, ampicillin, azactam, tobramycin, clindamycin and gentamycin; immunosuppressants; antiviral substances such as substances effective against hepatitis; enzyme inhibitors; hormones; neurotoxins; opioids; anti-protozoal compounds; modulators of cell-extracellular matrix interactions including cell growth inhibitors and antiadhesion molecules; inhibitors of DNA, RNA, or protein synthesis; anti-angiogenic factors; angiogenic factors; anti-secretory factors; anticoagulants and/or antithrombotic agents; co-factors for protein synthesis; endocrine tissue or tissue fragments; enzymes such as alkaline phosphatase, collagenase, peptidases, oxidases, etc.; polymer cell scaffolds with parenchymal cells; collagen lattices; cytoskeletal agents; hydroxyappetite; cartilage fragments; living cells such as chondrocytes, bone marrow cells, mesenchymal stem cells; natural extracts; genetically engineered living cells or otherwise modified living cells; expanded or cultured cells; tissue transplants; autogenous tissues such as blood, serum, soft tissue, bone marrow, etc.; bioadhesives; bone morphogenic proteins (BMPs, e.g., BMP-2); osteoinductive factor (IFO); fibronectin (FN); endothelial cell growth factor (ECGF); vascular endothelial growth factor (VEGF); human growth hormone (HGH); animal growth hormones; epidermal growth factor (EGF); interleukins, e.g., interleukin-1 (IL-1), interleukin-2 (IL-2); human alpha thrombin; transforming growth factor (TGF-beta); insulin-like growth factors (IGF-1, IGF-2); parathyroid hormone (PTH); platelet derived growth factors (PDGF); fibroblast growth factors (FGF, BFGF, etc.); periodontal ligament chemotactic factor (PDLGF); enamel matrix proteins; growth and differentiation factors (GDF, e.g., GDF-5), small peptides derived from growth factors above; bone promoters; cytokines; somatotropin; and nucleic acids or any combinations thereof.

In yet another embodiment, the implant of the present invention contain constructive adjuvant that may include metallic powder, osteogenic polymer, bone powder, collagen powder, radiographic powder, contrast and imaging agents, and mineralized powder.

In yet another aspect of the present invention, methods of reducing post-surgical infection are described. According to this aspect of the invention, methods follow such steps as identifying a patient in need of bone repair, and introducing an implant in the region in need of bone repair, wherein the implant comprising a polymeric/metallic body corresponding to an anatomical structure having plurality of microtubules, microchannels and reservoirs, wherein the polymeric body comprises a biocompatible and a bioresorbable polymer. In one embodiment, the reservoirs are interspersed in the matrix body of the implant. In another embodiment, the microtubules, the microchannels and the reservoir infrastructure provide a controlled release of the therapeutically active agent to the site of interest to facilitate and expedite tissue healing. In another embodiment, the reservoirs are of such polymeric material that allows refiling of the active ingredient even after the reservoir is depleted from its content.

In one preferred embodiment, the reservoir contains mechanism that minimizes or inhibits retrograde influx of fluid back into the reservoir. In a more preferred embodiment, the reservoir is of polymeric materials that are biodegradable. In another embodiment, the reservoir contains external access for filling the reservoir with a desired therapeutic composition to facilitate treatment or irrigation of the site.

In yet another embodiment, the presently described methods employ polymeric body that comprise a shaft region having one or more holes for receiving bone fasteners; and a head region extending from the shaft region and having a plurality of holes for receiving bone fasteners. In yet another embodiment, the presently described method employs implants that contain or are infused by therapeutic agents selected from the group consisting of antibiotics, anabolic steroids, analgesics, antihistamines, antibacterial agents, antifungal agents, bisphosphonates, anticoagulants, antineoplastics, antiparasitics, antipyretics, antispasmodics, antiviral agents, anti-uricemic agents, blood glucose-lowering agents, chemotherapeutic agents, erythropoietic drugs, growth regulators, hormone replacement agents, mineral supplements, narcotics, neuromuscular drugs, non-steroidal anti-inflammatories (NSAIDs), nutritional additives, therapeutic polypeptides, prostaglandins, steroids, uterine relaxants, vasoconstrictors, vasodilators, vitamins, wound healing agents, and combinations thereof. In another embodiment, the method employs implants that contains constructive adjuvant such as metallic powder, osteogenic polymer, bone powder, collagen powder, radiographic powder, contrast agents, hydroxyl appetite powder, fumed silica, colloidal silica, amorphous silica, quartz, alumina silicate, barium silicate glass, fluorosilicate glass, zirconia, calcium oxides, hydroxyapatites, titania, calcium phosphate, graphene oxide, and any combinations thereof.

In at least one embodiment, therapeutic agents may be incorporated into the core material of a core-shell polymeric filament by dry-blending together a selected antibiotic with a selected polymer to produce a master blend of an therapeutic agent-containing core material. For example, an antibiotic compounds and/or bone-growth-promoting compounds may be incorporated into the shell material of a core-shell polymeric filament by dry-blending together a selected antibiotic with a selected polymer to produce a master blend of an antibiotic-containing shell material.

In such embodiment, a core-shell polymeric filament comprising an antibiotic and/or a bone-growth-promoting compound in its core is prepared by combining the antibiotic-containing core material with a shell material that is absent any antibiotics or bone growth-promoting compounds. A master blend of core-shell polymeric filament comprising an antibiotic and/or a bone-growth-promoting compound in its shell is prepared by combining the antibiotic-containing shell material with a core material that is absent any antibiotics or bone growth-promoting compounds. The master blends comprising the antibiotic compositions and/or the bone-growth-promoting compounds should have a therapeutically effective amount of the antibiotic compositions and/or the bone-growth-promoting compounds to enable their deposition in the core components and the shell components of the polymeric filaments at rates that will provide a controlled release of such therapeutic agent at the site of implant.

In at least one embodiment, the concentration of the active therapeutic agent ranges from about 0.01% w/w to about 50% w/w of the therapeutic agent by weight of the total weight of the implant or prosthesis. This amount include for example, about 0.01% w/w, about 0.05% w/w, about 0.1% w/w, about 0.3% w/w, about 0.5% w/w, about 0.75% w/w, about 1.0% w/w, about 1.5% w/w, about 2.0% w/w, about 2.5% w/w, about 3.0% w/w, about 4.0% w/w, about 4.5% w/w, about 5.0% w/w, about 5.5% w/w, about 6.0% w/w, about 7.0% w/w, about 8.0% w/w, about 9.0% w/w, about 10.0% w/w, about 15.0% w/w, about 20.0% w/w, about 25.0% w/w, about 30% w/w, about 40% w/w, about 50% w/w and there between.

In another embodiment, the presently methods of making accounts for a step of incorporating a reservoir geometry and/or a microtubular channel system in said CAD model so designed to facilitate storage and delivery of suitable therapeutically active agent. In at least one embodiment, the implant is a copy of the patient's anatomical structure from the patient's sample. In another embodiment, the shape and size of the implant is provided based on the population standard customized for the age, weight and the race of the patient. In at least one embodiment, the polymer is PLA or PLA in combination with a secondary polymer selected from the group consisting of polyamides, polyesters, polycaprolactone (PCL), polydioxanone (PDX), and the like. polyglycolide-co-caprolactone, polyethylene oxide (PEO), polypropylene oxide (PPO), polyglycolide-co-trimethylene carbonate (PGA-co-TMC), poly(lactic-co-glycolic acid) (PLGA), polyglycolic acid (PGA), poly-L-lactide (PLLA), polyethylene glycol (PEG), polypropylene (PP), polyethylene (PE), polyetheretherketones (PEEK), poly(ester-ether), poly(L-leucine), poly(L-lysine), poly(amino acids), glycosaminoglycans (GAG), and polymethylmethacrylate (PMMA).

In yet another embodiment, the present methods employs metals selected from the group consisting of titanium, stainless, steel, cobalt, chrome and any combinations thereof. In yet another embodiment, the described methods provides for a polymeric body that further comprises a therapeutically active agent, a constructive adjuvant-, an osteogenic biologics or any combinations thereof.

In yet another aspect of the present invention, an individualized surgical kit is described containing a plurality of components comprising a patient-specific implant comprising a body corresponding to an anatomical structure having plurality of microtubules, wherein the body comprises a biocompatible material. According to one embodiment, the described kit provides implants that are of polymeric or metallic material in accordance to the preceding paragraphs. In a more preferred embodiment, the polymeric material is PLA.

In yet another aspect of the present invention, an automated system for customizing a patient specific implant may include a computer having a processor, a database storing one or more image templates, each template having a customizable region, wherein the customizable region includes an image of an anatomical model. In one embodiment, the database may reside locally in the computer. In another embodiment, the database may be in a remote location and accessible to the automated system via a communication link. The system may also include non-transitory computer readable memory coupled to the processor and containing programming instructions that will cause the computer to (a) obtain an anatomical model developed in a computer aided design (CAD) system; (b) customize the anatomical model per patient specific parameters using one or more image templates in the database and create a virtual image of the anatomical model; (c) incorporate at least one microchannel geometry within the anatomical model; (d) adjust the density infill to a measurement per patient specific parameters; (e) use 3D printing to form the implantable device based on the anatomical model. In one embodiment, the measurement range for density infill may be between 5 to 100%. In another embodiment, the 3D printing may include printing layer by layer.

Embodiments of the present invention provide apparatuses and methods for controlling the size and specificity of three-dimensional anatomical model-printing systems. A printing system, according to some embodiments of the present invention, may include a printing apparatus to print three-dimensional objects; a controller that may prepare the digital data that characterizes the 3-D object for printing, and control the operation of the printing apparatus; and a printing tray with a selected adhesion characteristic.

In another aspect, the final implant may be coated to alleviate tissue rejection and future graft-host tissue complications. In at least one embodiment, coating such as graphene oxide may be employed to cover the surface of the implant.

In a more preferred embodiment, the present implants that are made of bioresorbable PLA having a body that contains a reservoir and a microchannel network to facilitate the delivery and controlled elution of a therapeutically active agent. In an alternative embodiment, a reservoir may be utilized to contain a much larger dose of active agent as compared to, for example, the filament or microchannel containing configurations. In one embodiment, the therapeutic active agent is an antibiotic that provides coverage for colonies of anaerobic, gram negative, gram positive bacterial or any combinations of such bacterial. Such antibiotics include but are not limited to gentamicin, tobramycin, kanamycin, neomycin, ampicillin, methicillin, nafcillin, oxacillin, penicillin, ticarcillin, ciprofloxacin, vancomycin, cefazolin, cefepime, cefcdrioxone, clindamycin, aztreonom, imipenem, quniupristin/dalfopristin, chloramphenicol, doxycycline, metronidazole, nitrofurantoin, polymycin B, tetracyclines, biomycin, chloromycetin, streptomycins, azactam any pharmaceutically acceptable salts thereof and combinations thereof.

In one embodiment, the strength and stiffness of PLA can be appropriately controlled to eliminate the possibility of stress shielding unlike metallic implants.

In yet another aspect of the present invention, the described surgical implants follow different models, whether in shape, design, size, bulk, composition and so on, and these differences may be affected by different factors during the printing process, such as heat, chemical reactions of the photopolymer material to curing, internal strains (e.g., within the object) due to strains such as, for example shrinkage of the materials during curing and/or cooling, environmental influences within the printing apparatus, for example temperature fluctuations etc. Nevertheless, any such factors can further be determined to prepare a customized implant suitable for specific patient parameters.

By way of illustration, the following examples are given for the preparation of devices according to the present invention. However, these are for purposes of illustration and are not intended in any way to limit the scope of the invention.

### EXAMPLES

### Method/Experimental Procedure

In at least one embodiment, the present invention addresses the needs in orthopedic trauma patients who suffer open fractures and patients undergoing joint replacement surgery (hips and knees) and are at high risk for developing postoperative surgical infections.

The design and making of ‘smart’ 3D printed implants use PLA instead of traditionally employed PMMA. The mechanical properties, such as the strength and stiffness of PLA can be precisely controlled by varying the percentage of infill during 3D printing. The presently claimed implant material will be bioresorbable. Additionally, controlled drug delivery is possible when the implant design incorporated microchannels to facilitate sustained and consistent drug elution for 6-8 weeks to allow full dose elution.

According to various embodiments of designing and making of implantable devices described in this document, the printed bioresorbable large fragment plates can provide structural support during healing and have drug eluting properties. This is in contrast to metal plates which require full removal as they cannot elute antibiotics and is non-bioresorbable. In one embodiment, knee implants and intramedullary rods are developed. These specimens were prepared following the ASTM D638-10 tensile standards.

With reference to FIGS. 1A-1B, in one aspect, the present invention is directed to a medical implant made of suitable metallic or polymeric material. In one embodiment, implants are described that comprise a body corresponding to an anatomical structure, such as a femur plate 105, may have one or more microtubules 102, wherein the body comprises a biocompatible material. In another embodiment, the body of such implant is of polymeric or metallic material in up to 95% porous 103. In one embodiment, the present implant contains a body that is made of polymeric material comprising a melt processable polymer derived from a biodegradable, bioresorbable polymer.

In one embodiment, the percentage infill may be in the ranges of 25-55%. In one embodiment, the porous matrix forms a skeletal matrix 103 using natural PLA (15% infill) may be used to facilitate osteoblast recruitment and/or any other suitable connective tissue precursors to further expedite tissue healing and regeneration.

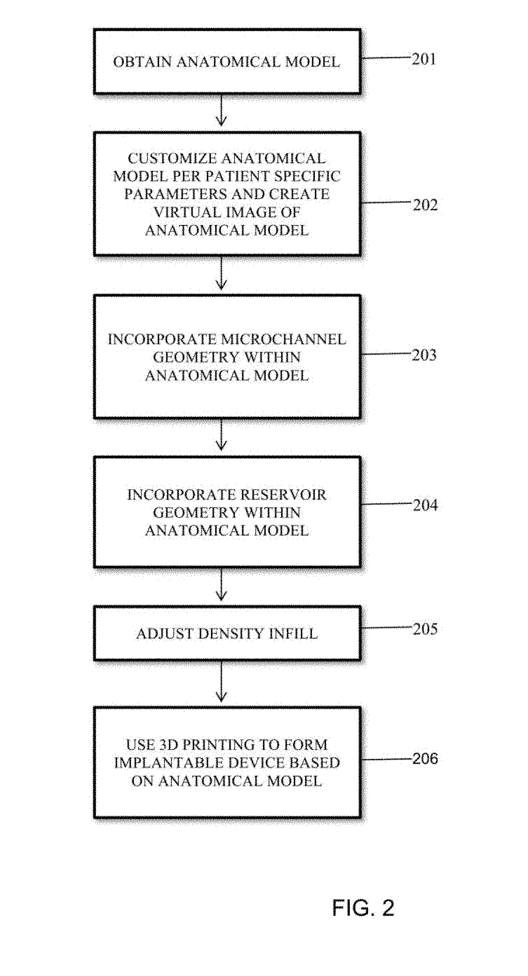

With reference to FIG. 2, according to one aspect of the present invention, methods of making an implantable device may include obtaining an anatomical model 201, customizing the anatomical model per patient specific parameters and create a virtual image of the anatomical model 202, incorporating at least one microchannel geometry within said anatomical model 203, adjusting the density infill to a measurement per patient specific parameters 205, and using three-dimensional printing to form the implantable device based on the anatomical model 206.

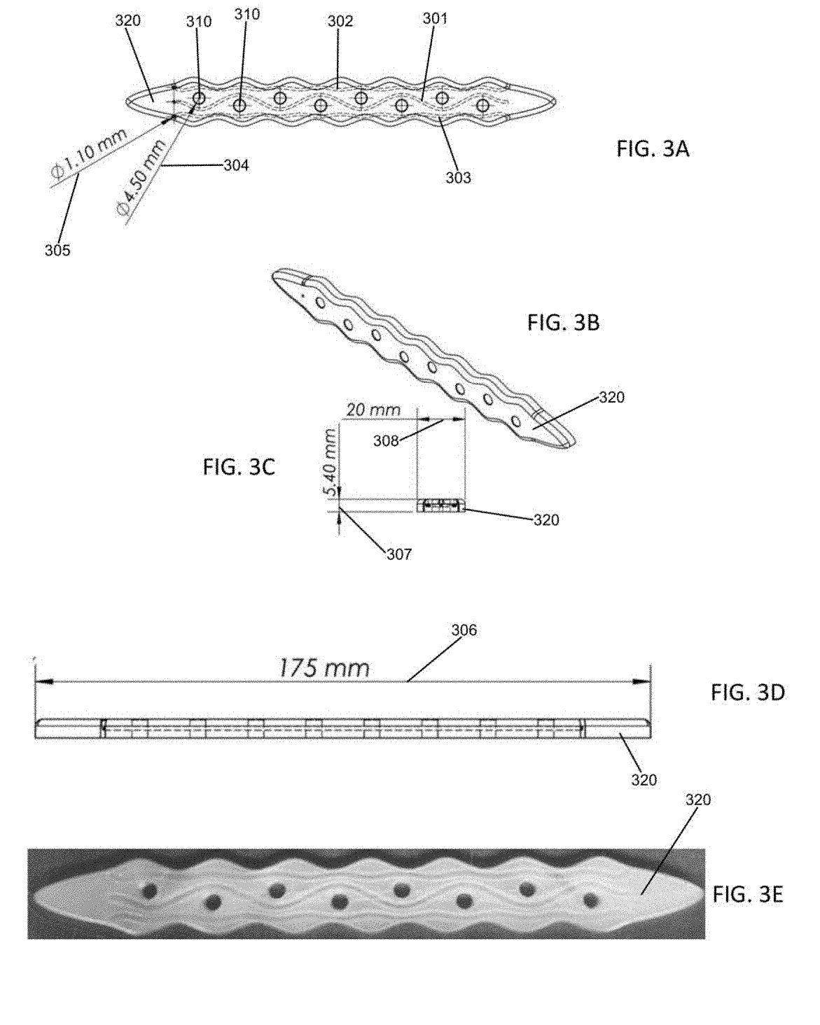

In one embodiment, the anatomical model may be developed in a computer aided design (CAD) system. In another embodiment, fused deposition modeling (FDM) techniques is employed to manufacture the implantable device. In one embodiment, the method of making the implantable device may also include incorporating at least one reservoir geometry within the anatomical model 204. In another embodiment, adjusting the density infill 205 may include adjusting the density infill to a measurement ranging between 5 to 100% per patient specific parameters. In another embodiment, the method of using three-dimensional printing to form the implantable device 206 may include developing the implantable device layer by layer. In one embodiment, the anatomical model is obtained by scanning a patient anatomy using a scanner. In another embodiment, the anatomical model is of polymeric or metallic material and the developing of the polymeric model in the computer aided design (CAD) system is for direct readability into a three-dimensional printing machine. These features are further illustrated by way of examples with reference to FIGS. 3-10.

In FIGS. 3A-3E, the design and making of a femur plate implant are illustrated. In one embodiment, the anatomical model of the femur plate 320 (FIGS. 3A-3D) may be developed from CAD system, and may include one or more holes 310 for receiving screw fasteners and one or more microchannels 301-303 for drug delivery. In one embodiment, the anatomic model may be customized per patient parameters. For example, the diameter of the one or more holes 310 may be in the range of 3-6 mm, 4-5 mm, and preferably 4.5 mm. In an embodiment, the femur plate may contain one or more microchannels 301-303 which extend along the outer edge of the femur plate 302, 303 or in a snake shape weaving between the one or more holes 301. The microchannels may be formed as grooves on the surface of the femur plate 320. In one embodiment, the diameter of the groove may be in the range from 0.2 mm to 2.5 mm, 0.8 mm to 1.5 mm and preferably 1.1 mm. In one embodiment, the femur plate may further carry one or multiple reservoirs to facilitate proper fluid flow from reservoirs within microchannel.

In one embodiment, the length 306 of the femur plate 320 may depend on the capacity of the 3D printer. For example, the length of the femur plate can be in the range of 80 mm to 510 mm, 100 mm to 300 mm, 150 mm to 250 mm, including 170 mm, 175 mm and 180 mm.

In one embodiment, the width of the femur plate 308 may be customized to be in the range from 10 mm to 35 mm, 15 mm to 25 mm and preferably 20 mm. In one embodiment, the thickness 307 of the femur plate implant may be between 3.0 mm and 6.0 mm, 4.5 mm to 5.5 mm, such as 5.4 mm. Based on the described anatomical model, the femur plate implant can be formed using three-dimensional printing and the method of making described in embodiments in FIG. 2, and the 3D printed device is shown in FIG. 3E.

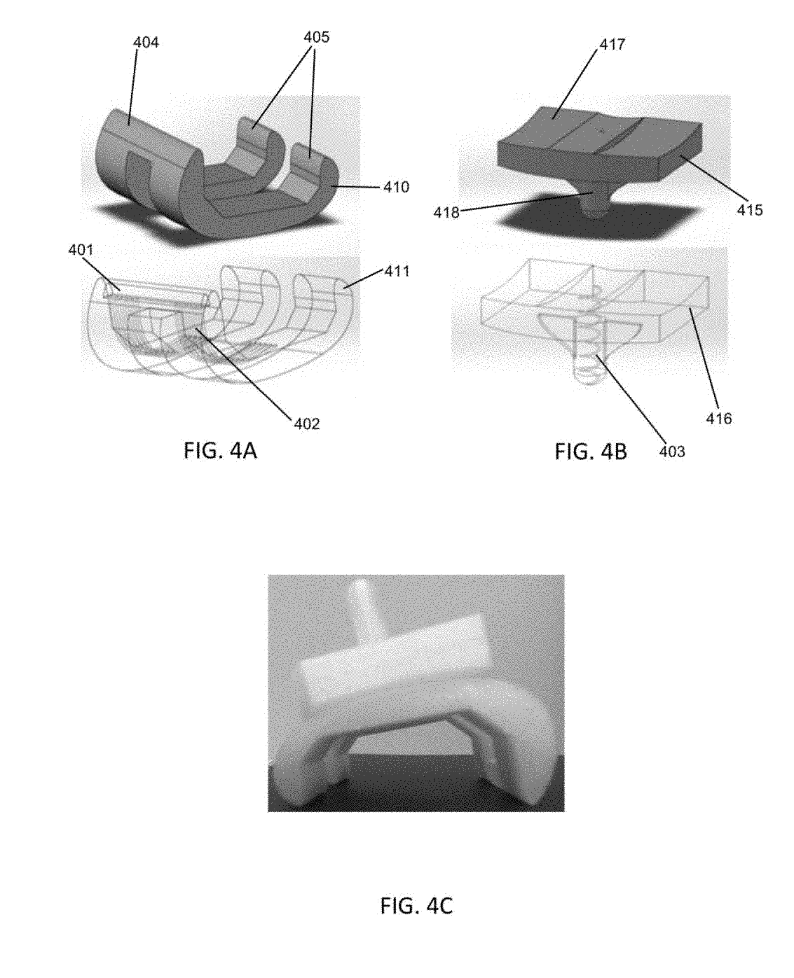

In FIGS. 4A-4B, the design and making of a knee implant are illustrated. In one embodiment, the anatomical model of the knee implant may have two pieces, the femoral component 410 and the tibial component 415. Their anatomical models, 411 and 416, each contains a reservoir and/or a microchannel. For example, the femoral component 411 may contain a reservoir 401 within a lateral member 404 that joins two longitudinal members 405, which are designed to be placed on a surface of the tibial component 415. The femoral component 411 may further contain multiple microchannels 402, which run longitudinally along each of the longitudinal members 405 and connect to the reservoir 401 to facility delivering the drug that is stored in the reservoir.

In another embodiment, the tibial component 416 may contain a flat surface 417 on which the femoral component 410 is to be placed, and a tibial insert 418 that is designed to be inserted into the tibia. In one embodiment, the tibial insert may contain a microchannel 403 that runs inside the insert and also extends out to the flat surface of the tibial component, permitting the drug to be delivered from the flat surface 417 through the microchannel 403 to the tibia. In one embodiment, the microchannel 403 can be of a spiral shape. In another embodiment, the microchannel can be of other curved tunnel or a straight tunnel. The 3D printed knee implant based on the described anatomical model and using the method described in embodiments in FIG. 2, is shown in FIG. 4C.

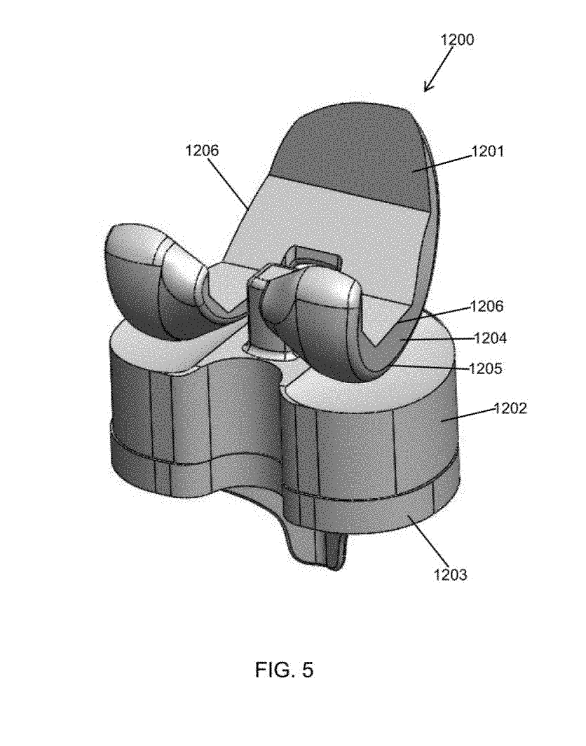

With reference to FIG. 5, according to one embodiment, the design and making of a total knee replacement implant are illustrated. The total knee replacement 1200 may include a femoral component 1201, a tibial component 1203 and a spacer 1202 that is placed in between the femoral and tibial components. In one embodiment, the femoral component may have two lateral surfaces, one on each opposite side 1204 (opposite side not shown), each lateral surface having an outer edge 1205, and inner edge 1206. The outer edge 1205 may be of a shape of semi-wheel configured to be placed on top of the spacer 1202. The inner edge 1206 may be formed by a multiple concatenated straight lines at different lengths. The outer edge and inner edge define the area of the lateral surface.

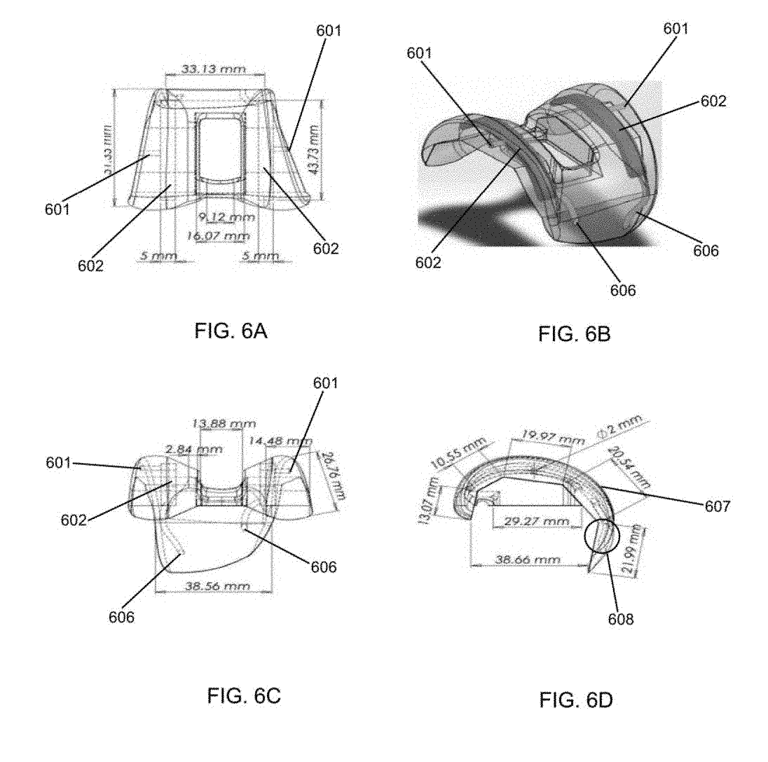

With reference to FIGS. 6A-6D, the anatomical model of the femoral component is further illustrated in detail. In one embodiment, the femoral component may contain a pair of two reservoirs 602, each reservoir being disposed inside the femoral component proximate to the lateral surface and between the outer edge and inner edge of the surface. In one embodiment, each reservoir may form a curve to correspond to the shape of the outer or inner edge. In one embodiment, each reservoir may be connected to two microchannels 601, 606, to facility drug delivery. In one embodiment, one of the two microchannels 601 may be connected to the reservoir at approximate a mid-segment point of the reservoir and extends outward perpendicular to and outside the lateral surface. The other microchannel 606 may be connected to the reservoir proximate to an end of the reservoir, extends from the end of the reservoir, travels along the curvature of the outer edge 607 (FIG. 6D) until it comes out the surface of the femoral component 608 (FIG. 6D).

The dimensions of the femoral component can be customized to patient specifications. For example, the diameter of the microchannel 601 that extends from the mid-segment point of the reservoir may be from 1.5 to 2.5 mm, and may be 2 mm. In another embodiment, the lateral dimension or the width of the reservoir in the lateral direction of the femoral component may be from 3 to 7 mm, and may be 5 mm.

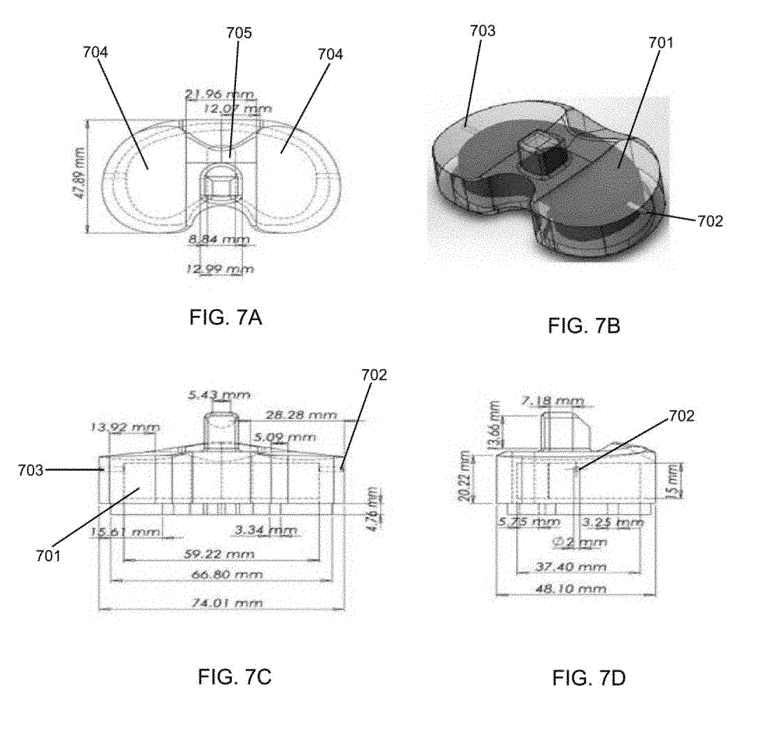

With reference to FIGS. 7A-7D, the anatomical model of the spacer of the total knee replacement is further illustrated in detail. In one embodiment, the spacer may contain a pair of two bean shape body 704 joining at a middle section 705. The spacer may have a height large enough to contain a reservoir chamber 701 inside the body. In one embodiment, the reservoir may take up a substantial space inside the spacer so that the reservoir forms a similar shape as the spacer body. In one embodiment, the reservoir may connect to two microchannels 702, 703, each at a distal point away from the middle section where the two bean shape body of the spacer meet, at opposite directions.

The dimensions of the spacer can be customized to patient specifications. For example, the length of the reservoir chamber may be in the range from 50-85 mm, preferably below 75 mm, and in one embodiment may be 59.22 mm. In one embodiment, the height or depth of the reservoir chamber may be in the range from 10-17 mm, 12 mm and may be 15 mm. In another embodiment, the diameter of the microchannels may be between 0.5 mm and 5 mm, 1 mm to 3 mm and may be 2 mm.

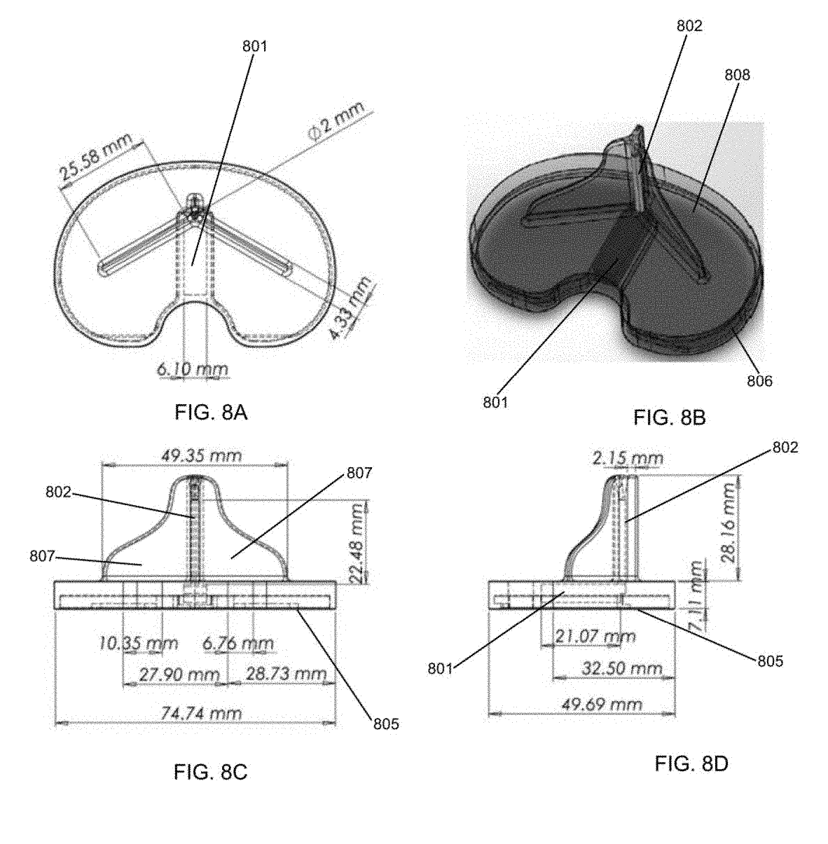

With reference to FIGS. 8A-8D, the anatomical model of the tibial component of the total knee replacement is further illustrated in detail. In one embodiment, the tibial component may contain a body 806 that is of substantially the same shape as the spacer body and configured to be disposed between the spacer and the tibia. In one embodiment, the tibial component body has a top surface 805 that is designed to touch a surface of the spacer while the tibial component is seated between the tibia and the spacer. In one embodiment, the tibial component body has another surface 808 at opposite side of the top surface, and the tibial component may contain an insert extending perpendicularly from the opposite surface 808 and configured to be inserted into the tibia when it is disposed between the tibia and the spacer. In one embodiment, the insert may contain a pair of two plates 807 joining together and forming an angle, where the joint forms a seam perpendicular to the opposite surface 808.

In one embodiment, the tibial component may contain a reservoir 801 inside the body at the middle section. Additionally, the tibial component may also contain a microchannel 802 along the seam where the two insert plates 807 join so that the microchannel 802 connects to the reservoir at one end and extends substantially perpendicular to the opposite surface 808. The dimensions of the tibial component can be customized to patient specifications. For example, the length of the reservoir may be in the range from 18-22 mm, and may be 21.07 mm. In another embodiment the diameter or width of the reservoir may be in the range from 5-7 mm, and may be 6.10 mm. In another embodiment, the microchannel connecting the reservoir may run the length between 25 and 30 mm, and may be 28.16 mm in length.

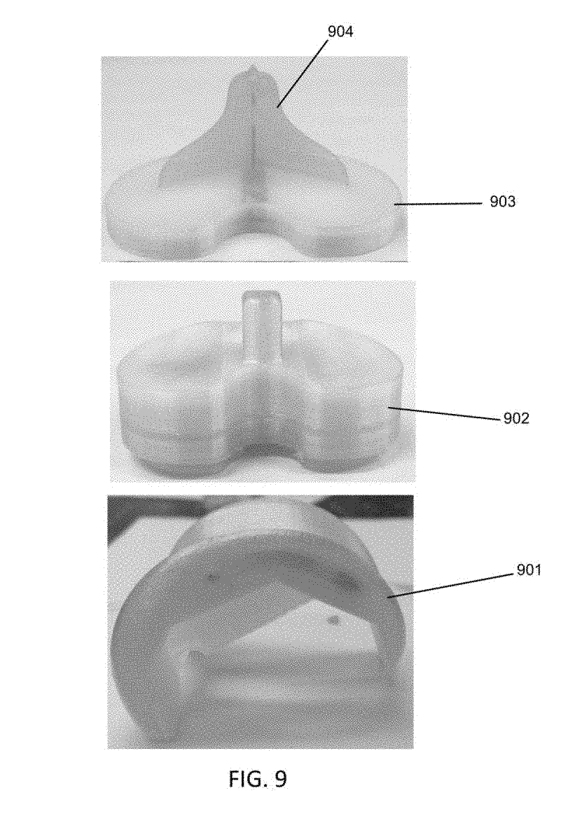

With reference to FIG. 9, the 3D printed total knee replacement implant 900 is developed based on the described anatomical model described in FIGS. 6-8 and using the method described in embodiments in FIG. 2. As shown in FIG. 9, the 3D printed total knee replacement 900 contains the femoral component 901 placed on a surface of the spacer 902, which is disposed between the femoral component 901 and the tibial component 903. The tibial component 903 further includes an insert 904 configured to be inserted into the tibia when the tibial component is placed on the tibia. Other various implantable devices may be designed and made according to the embodiment described in FIG. 2.

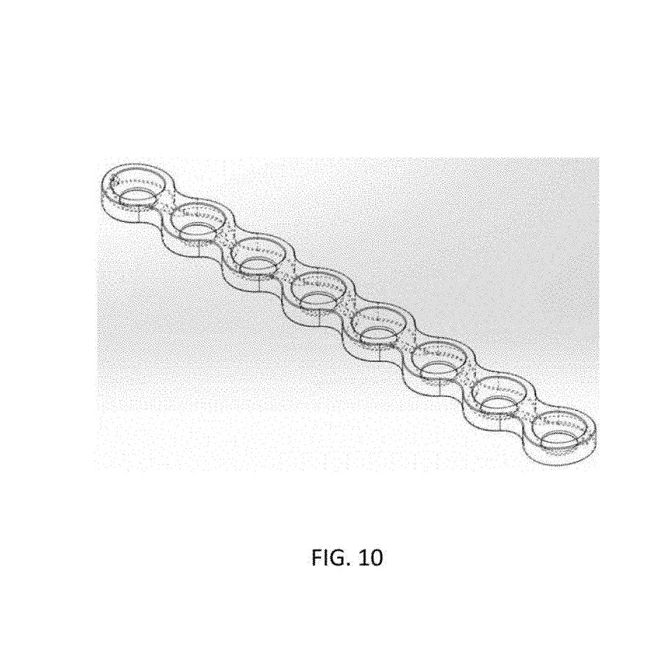

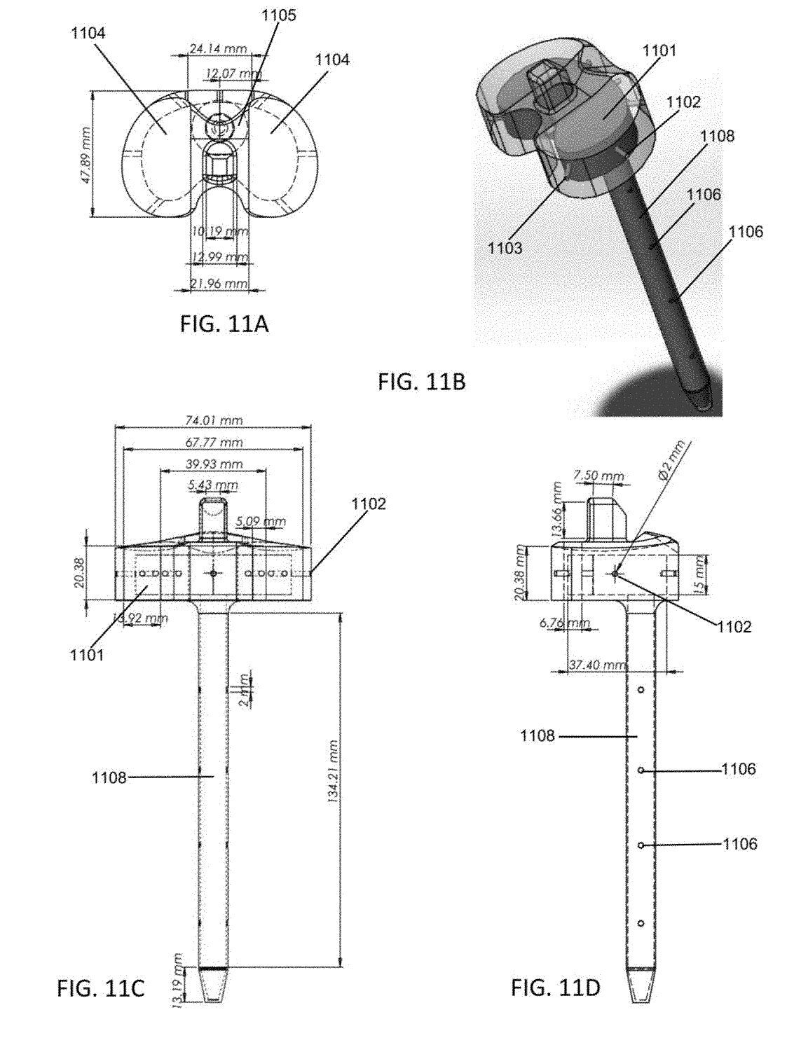

For example, an anatomic model of a pelvic plate is shown in FIG. 10, and a redesigned plastic liner with reservoir and microchannels is shown in FIG. 11. With reference to FIGS. 11A-11D, the plastic liner may contain a pair of two bean shape body 1104 joining at a middle section 1105. The plastic liner may have a height large enough to contain a reservoir chamber 1101 inside the body. In one embodiment, the reservoir may take up a substantial space inside the liner so that the reservoir forms a similar shape as the liner body. In one embodiment, the reservoir may connect to multiple microchannels 1102, 1103, each at a distal point away from the middle section where the two bean shape body of the liner meet, at opposite directions. In an embodiment, the liner body may extend a stem, where the stem has multiple holes 1106 on the stem wall for drug delivery.

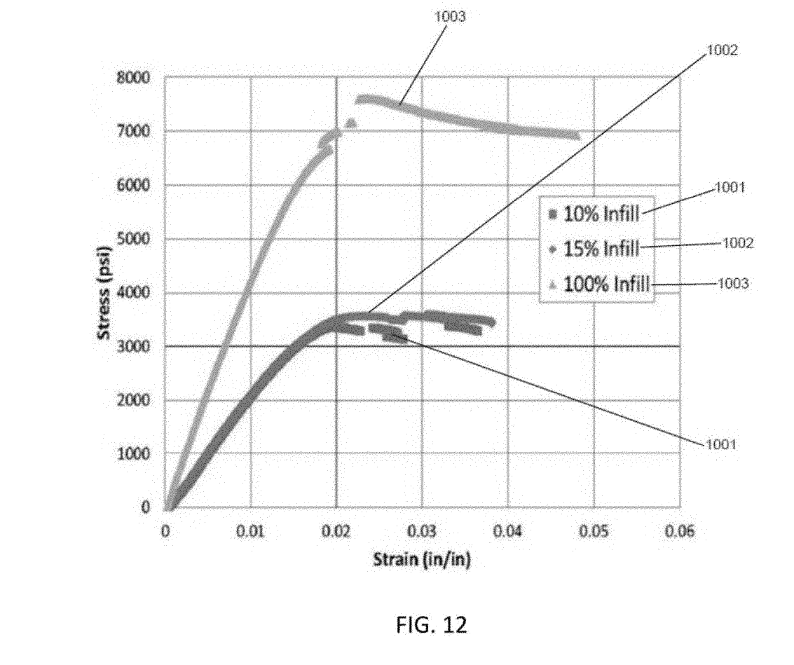

With reference to FIG. 12, the tensile tests performed determine the ultimate strength and modulus for printed PLA as a function of infill percentage and strain rate. These preliminary results indicate that by controlling the infill percentage, desired strength/stiffness for the implant can be achieved.

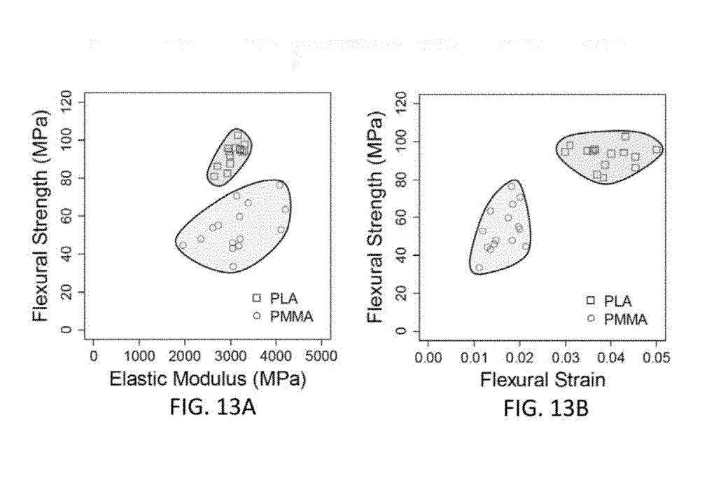

With reference to FIGS. 13A-13B, various strength versus modulus plots are shown for PLA and PMMA, and less scatter in the mechanical property of printed PLA can be observed when compared to PMMA (current gold standard for infection treatment).

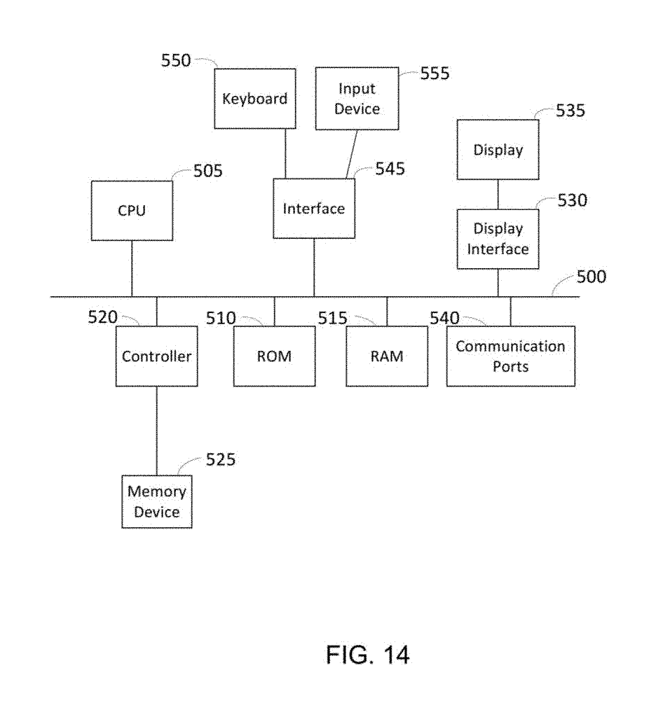

FIG. 14 depicts an example of internal hardware that may be included in any of the electronic components of the system, the automated system for developing or customizing the anatomic model, the printing system or other computer systems. An electrical bus 500 serves as an information highway interconnecting the other illustrated components of the hardware. Processor 505 is a central processing device of the device, configured to perform calculations and logic operations required to execute programming instructions. As used in this document and in the claims, the terms “processor” and “processing device” may refer to a single processor or any number of processors or processor cores in one or more processors. The device may include read only memory (ROM) 510, random access memory (RAM) 515, or other types of memory devices 525, such as flash memory, hard drives and other devices capable of storing electronic data. A memory device may include a single device or a collection of devices across which data and/or instructions are stored.

An optional display interface 530 may permit information from the bus 500 to be displayed on a display device 535 in visual, graphic or alphanumeric format. An audio interface and audio output (such as a speaker) also may be provided. Communication with external devices may occur using various communication ports or devices 540 such as a portable memory device reader/writer, a transmitter and/or receiver, an antenna, an RFID tag and/or short-range or near-field communication circuitry. The communication device 540 may be attached to a communication network or a communication link, such as the Internet, a local area network or a cellular telephone data network.

The hardware may also include a user interface sensor 545 that allows for receipt of data from input devices 555 such as a keyboard 550, a mouse, a joystick, a touchscreen, a remote control, a pointing device, a video input device (camera) and/or an audio input device (microphone). Various methods of activation, validation and/or authorization described in this document may be performed by the central processing device 505 or a controller 520.

### Example 1

### Coating PLA Beads with Antibiotics

In this example, bioactive 3D printing filaments using gentamicin sulfate, tobramycin, and nitrofurantoin antibiotics were created and a process was developed to coat PLA beads with 1%, 2.5%, and 5% coating of the antibiotics. The coated PLA beads were then extruded into filament usable for FDM 3D printing. The 3D printing material used in the additive coating study were commercially-available PLA beads. To perform the coating process, the antibiotics chosen for testing were gentamicin, tobramycin, and nitrofurantoin, with KJL 705 Silicone Oil as the chemical used to hold the antibiotics to the heads.

During the coating portion of the experiment, basic chemical measuring devices, test tubes, and sterilization chemicals were required. A vortex machine to ensure complete coating, as well as a mortar and pestle to crush the antibiotics into a uniform powder, were also used. For extrusion process, an Extrusionbot filament extruder turned the PLA beads into printable filament. To 3D print the objects tested, a Makerbot Replicator 5th generation was chosen, along with the Makerware software program for converting computer models to the file type used by the printer.

In order to coat the PLA beads with the antibiotics, the beads were placed in disposable, sterile test tubes. They were then covered with silicone oil and vortexed in order to ensure complete coating across each bead. Batches of beads weighed 20 grams, and 15 pL of oil were used. The amount of oil used is important because too much oil will lead to bead clumping and extrusion flow problems later. After silicone oil, the beads were placed in a new container to prevent loss of additives on the surface of the container. Next, antibiotic additives were ground with a mortar and pestle to ensure uniform powder size. The additives were introduced to the beads from sterile and disposable plastic test tubes and vortexed again to coat the beads. The amounts of additives tested were 1%, 2.5%, and 5% weight addition. By this, it is meant that a 1% weight addition used 200 mg of powder, 2.5% used 500 mg, and 5% used 1 g of an additive. The individual breakdown for each antibiotic is as follows: gentamicin was used to coat pellets at 1%, 2.5% and 5%; tobramycin was used to coat pellets at 1% and 2.5%; nitrofurantoin was used to create coatings at 1%.

The maximum amount used for the first layer of coating was only 5% because any more additives would have fallen off the beads. This coating process can be repeated to cover beads with another additive layer. Multiple tests were done to determine how many layers of additive were possible before extrusion was no longer possible. After the bead coating process, the beads were put through a filament extruder to create filament for printing. The filament was stored in sterile bags and refrigerated until printing so that they remained unsoiled. In the last step the filament were loaded into the 3D printer and printing various shapes for testing. The temperature setting on the printer was set to 220° Celsius, which is standard for PLA, and a resolution setting of 50 microns.

For the coating process, to increase the coating to greater percentages, the process can be repeated multiple times, up to a coating of 25%. After this percentage, the extrusion machine continuously jammed and could not create filament.

To determine the effectiveness of the PLA bead coating with antibiotics, the samples were tested for antimicrobial activity by both agar diffusion and liquid nutrient broth. It was found that through the manufacturing process, the compounds retained their antimicrobial or cell growth inhibiting properties, even though heat was involved in manufacturing. Using antibiotic-laden PMMA bone cement as a control volume, it was found that the bioactivity of the antibiotic-laden PLA filaments were equivalent, and in some cases superior, to that of the PMMA. According to this study, the feasibility of infusing antibiotics or other additives into a 3D-printable material.

### Example 2

### Total Knee Replacement CAD Development

Using information gathered on total knee replacement design aspects, bone geometry data provided by Mimics software, and a total knee replacement sample, a total knee replacement three dimensional model was developed in Solidworks. FIG. 5 shows the model iteration. The total knee replacement 1200 includes a femoral component 1201, a tibial component 1203 and a spacer 1202 that is placed in between the femoral and tibial components. The femoral component possesses two lateral surfaces, one on each opposite side 1204 (opposite side not shown), each lateral surface having an outer edge 1205, and inner edge 1206. The outer edge 1205 may be of a shape of semi-wheel configured to be placed on top of the spacer 1202. The inner edge 1206 may be formed by a multiple concatenated straight lines at different lengths. The outer edge and inner edge define the area of the lateral surface.

### Example 3

### Use of Microchannel and Reservoir Network Inside the Orthopedic Implant

In this example drug delivery methods were developed for manufacturing a microchannel/reservoir network inside the orthopedic implant, filled with antibiotics, which would then elute through the implant, primarily via diffusion, for the 6-8 week treatment period.

FIGS. 6A-6D elaborates in at least one such embodiment. In this embodiment, femoral component contain a pair of two reservoirs 602, each reservoir being disposed inside the femoral component proximate to the lateral surface and between the outer edge and inner edge of the surface. Each reservoir form a curve to correspond to the shape of the outer or inner and is connected to two microchannels 601, 606, to facility drug delivery, one of which, 601, is connected to the reservoir at approximate a mid-segment point of the reservoir and extends outward perpendicular to and outside the lateral surface. In this example, the other microchannel 606 is connected to the reservoir proximate to an end of the reservoir, extends from the end of the reservoir, travels along the curvature of the outer edge 607 (FIG. 6D) until it comes out the surface of the femoral component 608 (FIG. 6D). Those of ordinary skill in the art can appreciate that these type of designs can be implemented in an orthopedic device, such as a spacer for second stage revision surgery of total knee replacements, and be manufactured using additive manufacturing for construction of the internal geometry.

### Example 4

### Femur Reconstruction Plate 3 Point Bend Testing

Applied to a bone, three-point bending occurs when three forces act on the specimen and create two equal moments. The moments correspond to the product of each support force and its distance normal to the axis of rotation. The axis of rotation is located at the loading point. Upon yield, a homogeneous and symmetrical bone should break at the site where the load is applied.

A common bone fracture caused by three-point bending is the “boot top” tibial fracture. This injury occurs when skiers fall over their ski boots, loading the tibia with the boot top and resulting in fracture. In this example, the load and crosshead displacement data are recorded during three-point bend tests, with load in the form of bending moment. This data can be used to determine bending moment, bending stress, breaking stress, modulus of elasticity, and strain. The formulas used to determine these values assume the test specimen is an isotropic, homogeneous, and linearly elastic material in beam form. Although whole bone does not align with each criterion, the formulas can be used to compare properties between studies.

In an example, ten PMMA and ten PLA bars will be loaded in a three-point bending apparatus. Following ASTM D790-10, loading will occur at midspan of the specimen, with span determined referencing the geometry of the specimen. Loading rate is determined through Equation 1

R=ZL²/6d (1)

where R is the rate of crosshead motion, L is span, d is depth of beam, and Z is straining of the outer fiber, which shall equal 0.01 mm/mm/min.

Elastic modulus will be determined using Equation 2

E_{B}=L³M/4bd³ (2)

where E_{B} is the modulus of elasticity in bending, L is the support span, b is the width of the beam, d is the depth of the beam, and m is the slope of the tangent to the initial straight-line portion of the load deflection curve.

Flexural strength will be determined with maximum flexural stress, using Equation 3

σ_{f}=PL/2bd² (3)

where σ_{f} is the stress in the outer fibers at the midpoint, P is maximum load on the load-deflection curve, L is span, b is width, and d is depth of the specimen.

Flexural strain will be determined using Equation 4

ε_{f}=6Dd/L² (4)

where ε_{f} is the strain in the outer surface, D is the maximum deflection of the center of the beam, L is span and d is depth.

Five femur plates per infill/orientation combination was printed in randomized order, all with 45 degree crosshatch infill pattern. Varying infill percentages as well as the orientations was used for printing. The infills and orientations of the specimens for this experiment provided the following results.

In summary, the 50%—90 degrees femur plates resulted in the lowest flexural strength when compared to plates of 25%, 50%, 75%, and 100%, each with orientation varying between 0 and 90 degrees. The 100%—0 degrees femur plates resulted in the highest flexural strength in the same experiment. The bars of 100% infill and Z orientation outperformed all other infill and orientation combinations (25%, 50%, 75%, 100% at 0 or 90 degrees) based on flexural strength. Therefore, the 100%—Z femur plates generate high values of bending stress for this experiment.

### Example 5

### Computational Fluid Dynamic Analysis

In this experiment dimensional analysis and numerical simulations were carried out to analyze fluid flow through micro channels. Dimensional analysis was performed to observe the physical phenomena of fluid flows at microscales. Model validation may be conducted on flat plates as well as circular pipes and the results were benchmarked with the analytical solution as described in the art. Transient simulations were carried out on select problems to model the flow through porous media.

### Dimensional Analysis

To perform this analysis, the dimensionless numbers were employed for analyzing the physical characteristics of microfluidic flows. These numbers are: i) Reynold's Number (Re) that compares the inertial forces with viscous forces; ii) Bond Number (Bo) that captures the importance of interfacial forces with respect to gravity; iii) Capillary Number (Ca) which expresses the relation between viscous forces and interfacial forces; iv) Knudsen Number (Kn) that defines the transition between micro and nano scales; v) Peclet number (Pe) which relates convective transport to diffusive transport of fluids.

Reynold's Number is defined in Equation 5:

Re=ρVD/μ (Eq. 5)

where ρ is the fluid density, V is fluid velocity, D is the Hydraulic Diameter and μ is the dynamic viscosity.

Bond Number is defined in Equation 6:

Bo=ΔρgD²/Tₛ (Eq. 6)

where, Δp is the density difference between the two fluids, g is acceleration due to gravity Tₛd is the surface tension.

Capillary Number is defined in Equation 7:

Ca=μV/Tₛ (Eq. 7)

Knudsen number is defined in Equation 8:

Kn=L_{mfp}/D (Eq. 8)

where, Lmfp is the mean free path (Lmfp˜molecular diameter for liquids).

Peclet number, which relates convection to diffusion in fluid transport, is shown in Equation 9:

Pe=VD/D_{f} (Eq. 9)

where D_{f} is the Diffusion coefficient of the solute in the solvent. Next, we will illustrate the dimensional analysis that was carried out for bone plates (femur). A reservoir of 10 mL is taken into consideration and the flow rate, Q is calculated over 6 weeks to be Q=2.756×10⁻¹² m³/sec

The input parameters can be defined as μ=0.001 Pa·s, ρ=1000 kg/m³ Ts=0.072 N/m, D_{f}=1.74×10⁻⁹ m²/S (Sodium Chloride in water).

The pressure drop (ΔP) was calculated using the Hagen Poiseuille Equation 10:

ΔP=8μVL/R² (Eq. 10)

where, L is the length of the tube and R is the radius of the tube. The dimensionless numbers that were obtained for microchannels with diameters 0.5 mm, 1 mm and 2 mm were calculated and shown in Table below:

| D | V = Q/A | dP (Pa) | Re | Bo | Ca | Kn | Pe |
|---|---|---|---|---|---|---|---|
| 0.5 | 1.4E−02 | 3.1E−01 | 7.0E−03 | 3.4E−02 | 1.9E−07 | 5.0E−07 | 4.0E+00 |
| 1 | 3.5E−03 | 2.0E−02 | 3.5E−03 | 1.4E−01 | 4.9E−08 | 2.5E−07 | 2.0E+00 |
| 2 | 8.8E−04 | 1.2E−03 | 1.8E−03 | 5.5E−01 | 1.2E−08 | 1.3E−07 | 1.0E+00 |

As such the following observations are made from Table: i) Creeping Flow (Re<<1); ii) Gravity and surface tension both can affect the flow; iii) Surface tension dominates the flow over viscous forces (Ca<0.00001); iv) Continuum Model and no slip boundary condition (Kn<0.001); v) Flow is driven by diffusion (Pe<1000). In view of these observations, the viscosity and concentration needed for a proper flow of a therapeutic agent containing fluid through the microchannels is achieved to provide controlled release of the active agent of choice.

By optimizing the geometry (reservoir size, reservoir shape, number of reservoirs and microchannels), material infill, the drug delivery can be precisely controlled and measured using a spectrophotometer or a high-performance liquid chromatography (HPLC) system as well as on the basis of bacterial kill studies.

While this invention has been described with an emphasis upon preferred embodiments, it will be obvious to those of ordinary skill in the art that variations in the preferred implant and methods can be used and that it is intended that the invention can be practiced otherwise than as specifically described herein. Accordingly, this invention includes all modifications encompassed within the spirit and scope of the invention as defined by the claims that follow.

1. An implant comprising a body corresponding to an anatomical structure having plurality of microtubules, wherein the body comprises a biocompatible material.

2. The implant of claim 1, wherein the body is of polymeric or metallic material.

3. The implant of claim 2, wherein the body is of polymeric material comprising a melt processable polymer derived from a biodegradable, bioresorbabler polymer.

4. The implant of claim 3, wherein the polymeric body comprise a juxta-articular and/or shaft region having one or more holes for receiving bone fasteners; and a head region extending from the shaft region and having a plurality of holes for receiving bone fasteners.

5. The implant of claim 1, wherein the body further comprising reservoirs and microchannels for storing and delivering a therapeutically active agent.

6. The implant of claim 1, wherein the body further comprising holes.

7. The implant of claim 6, wherein the holes are in the shape selected from the group consisting of circular, elliptical and any combinations thereof.

8. The implant of claim 7, wherein the holes are designed to accept locking and non-locking screws.

9. The implant of claim 8, wherein the circular holes are threaded or unthreaded screw holes having a diameter ranging from 0.1-5 cm.

10. The implant of claim 3, wherein the polymeric body further comprises a porous or solid matrix.

11. The implant of claim 10, wherein the polymeric density ranges from about 5 to 100% infill.

12. The implant of claim 3, wherein the polymeric material is selected from the group consisting of poly lactides (PLA), polyamides, polyesters, polycaprolactone (PCL), polydioxanone (PDX), and the like. polyglycolide-co-caprolactone, polyethylene oxide (PEO), polypropylene oxide (PPO), polyglycolide-co-trimethylene carbonate (PGA-co-TMC), poly(lactic-co-glycolic acid) (PLGA), polyglycolic acid (PGA), poly-L-lactide (PLLA), polyethylene glycol (PEG), polypropylene (PP), polyethylene (PE), polyetheretherketones (PEEK), poly(ester-ether), poly(L-leucine), poly(L-lysine), poly(amino acids), glycosaminoglycans (GAG) and combinations thereof.

13. The implant of claim 2, wherein the metal is selected from the group consisting of titanium, stainless, steel, cobalt, chrome and any combinations thereof.

14. The implant of claim 1 or 4, wherein the polymeric body further comprises a therapeutically active ingredient, a constructive adjuvant, an osteogenic biologics or any combinations thereof.

15. The implant of claim 14, wherein the active ingredients are selected from the group consisting of antibiotics, anabolic steroids, analgesics, antihistamines, anti-arrhythmia agents, antihypertensives, antiasthmatics, antibacterial agents, antifungal agents, anticonvulsants, anticoagulants, antihyperglycemic agents, anti-inflammatories, antineoplastics, antiparasitics, antipyretics, antispasmodics, antiviral agents, anti-uricemic agents, blood glucose-lowering agents, chemotherapeutic agents, cholesterol-reducing agents, coronary dilators, erythropoietic drugs, fungicides, growth regulators, hormone replacement agents, mineral supplements, narcotics, neuromuscular drugs, non-steroidal anti-inflammatories (NSAIDs), nutritional additives, peripheral vasodilators, therapeutic polypeptides, prostaglandins, anti-restless leg syndrome agents, steroids, uterine relaxants, vaginal preparations, vasoconstrictors, vasodilators, vitamins, wound healing agents, and combinations thereof.

16. The implant of claim 14, wherein the constructive adjuvant further is selected from the group consisting of metallic powder, osteogenic polymer, bone powder, collagen powder, radiographic powder, contrast agents, and mineralized powder.

17. A method of reducing post-surgical infection comprising the steps of identifying a patient in need of bone repair, and introducing an implant in the region in need of bone repair, wherein the implant comprising a polymeric/metallic body corresponding to an anatomical structure having plurality of microtubules, microchannels and reservoirs, wherein the polymeric body comprises a biocompatible and a bioresorbable polymer.

18. The method of claim 17, wherein the polymeric body comprise a shaft region having one or more holes for receiving bone fasteners; and a head region extending from the shaft region and having a plurality of holes for receiving bone fasteners.

19. The method of claim 18, wherein the holes are in the shape selected from the group consisting of circular, elliptical and any combinations thereof.

20. The implant of claim 19, wherein the holes are designed to accept locking and non-locking screws.

21. The method of claim 20, wherein the circular holes are threaded or unthreaded screw holes having a diameter ranging from 0.1-5 cm.

22. The method of claim 11, wherein the polymeric body further comprises a porous or solid matrix.

23. The method of claim 22, wherein the polymeric density ranges from about 5 to 100% infill.

24. The method of claim 23, wherein the polymer is selected from the group consisting of poly lactides (PLA), polyamides, polyesters, polycaprolactone (PCL), polydioxanone (PDX), and the like. polyglycolide-co-caprolactone, polyethylene oxide (PEO), polypropylene oxide (PPO), polyglycolide-co-trimethylene carbonate (PGA-co-TMC), poly(lactic-co-glycolic acid) (PLGA), polyglycolic acid (PGA), poly-L-lactide (PLLA), polyethylene glycol (PEG), polypropylene (PP), polyethylene (PE), polyetheretherketones (PEEK), poly(ester-ether), poly(L-leucine), poly(L-lysine), poly(amino acids), glycosaminoglycans (GAG) and combinations thereof.

25. The method of claim 17, wherein the polymeric body further comprises a therapeutically active ingredient, a constructive adjuvant, or an osteogenic biologics.

26. The method of claim 17, wherein the therapeutically active ingredients are selected from the group consisting of antibiotics, anabolic steroids, analgesics, antihistamines, anti-arrhythmia agents, antihypertensives, antiasthmatics, antibacterial agents, antifungal agents, bisphosphonates, anticonvulsants, anticoagulants, antihyperglycemic agents, antineoplastics, antiparasitics, antipyretics, antispasmodics, antiviral agents, anti-uricemic agents, blood glucose-lowering agents, chemotherapeutic agents, cholesterol-reducing agents, coronary dilators, erythropoietic drugs, growth regulators, hormone replacement agents, mineral supplements, narcotics, neuromuscular drugs, non-steroidal anti-inflammatories (NSAIDs), nutritional additives, therapeutic polypeptides, prostaglandins, steroids, uterine relaxants, vaginal preparations, vasoconstrictors, vasodilators, vitamins, wound healing agents, and combinations thereof.

27. The method of claim 18, wherein the constructive adjuvant further is selected from the group consisting of metallic powder, osteogenic polymer, bone powder, collagen powder, radiographic powder, contrast agents, hydroxyl appetite powder, fumed silica, colloidal silica, amorphous silica, quartz, alumina silicate, barium silicate glass, fluorosilicate glass, zirconia, calcium oxides, hydroxyapatites, titania, calcium phosphate, graphene oxide, and any combinations thereof.

28. A method of making an implantable device comprising the steps of (a) obtaining an anatomical model in a computer aided design (CAD) system; (b) customizing the anatomical model per patient specific parameters and creating a virtual image of the anatomical model; (c) incorporating at least one microchannel geometry within said anatomical model; (d) adjusting the density infill to a measurement in a range from 5% to 100% per patient specific parameters; (e) using 3D printing to form the implantable device based on said anatomical model.

29. The method of claim 28, wherein obtaining the anatomical model includes scanning a patient anatomy using a scanner.

30. The method of claim 29, wherein the anatomical model is of polymeric or metallic material.

31. The method of claim 29, wherein obtaining of the anatomical model developed in the computer aided design (CAD) system includes directly reading the anatomical model into a three-dimensional printing machine.

32. The method of claim 28, further comprising incorporating a reservoir geometry in said CAD model.

33. The method of claim 31, wherein the anatomic model is of polymeric material selected from the group consisting of selected from the group consisting of poly lactides (PLA), polyamides, polyesters, polycaprolactone (PCL), polydioxanone (PDX), and the like. polyglycolide-co-caprolactone, polyethylene oxide (PEO), polypropylene oxide (PPO), polyglycolide-co-trimethylene carbonate (PGA-co-TMC), poly(lactic-co-glycolic acid) (PLGA), polyglycolic acid (PGA), poly-L-lactide (PLLA), polyethylene glycol (PEG), polypropylene (PP), polyethylene (PE), polyetheretherketones (PEEK), poly(ester-ether), poly(L-leucine), poly(L-lysine), poly(amino acids), glycosaminoglycans (GAG) and combinations thereof.

34. The method of claim 33, wherein the polymer is PLA.

35. The method of claim 30, wherein the anatomic model is of metal material selected from the group consisting of titanium, stainless, steel, cobalt, chrome and any combinations thereof.

36. The method of claim 30, wherein the anatomic model is of polymeric material and the polymeric body further comprises a therapeutically active ingredient, a constructive adjuvant, an osteogenic biologics or any combinations thereof.

37. An individualized surgical kit containing a plurality of components comprising a patient-specific implant comprising a body corresponding to an anatomical structure having plurality of microtubules, wherein the body comprises a biocompatible material.

38. The kit of claim 37, wherein the body of the implant is of polymeric material comprising a melt processable polymer derived from a biodegradable, bioresorbabler polymer

39. The kit of claim 38, wherein the polymeric body corresponding to an anatomical structure having plurality of microtubules and/or microchannels and reservoirs, wherein the polymeric body comprises a biocompatible and a bioresorbable polymer.

40. The kit of claim 39, wherein the polymeric body further comprises a porous/solid matrix.

41. The kit of claim 39, wherein the polymer is selected from the group consisting of poly lactides (PLA), polyamides, polyesters, polycaprolactone (PCL), polydioxanone (PDX), and the like. polyglycolide-co-caprolactone, polyethylene oxide (PEO), polypropylene oxide (PPO), polyglycolide-co-trimethylene carbonate (PGA-co-TMC), poly(lactic-co-glycolic acid) (PLGA), polyglycolic acid (PGA), poly-L-lactide (PLLA), polyethylene glycol (PEG), polypropylene (PP), polyethylene (PE), polyetheretherketones (PEEK), poly(ester-ether), poly(L-leucine), poly(L-lysine), poly(amino acids), glycosaminoglycans (GAG) and combinations thereof.

42. A system for customizing a patient specific implant, comprising:
a processor;
a database containing one or more image templates, each template having a customizable region, wherein the customizable region includes an image of an anatomical model; and
a non-transitory computer readable memory coupled to the processor and containing programming instructions that, when executed, cause the processor to:
obtain an anatomical model developed in a computer aided design (CAD) system,
customize the anatomical model per patient specific parameters using one or more image templates in the database and create a virtual image of the anatomical model,
incorporate at least one microchannel geometry within said anatomical model, adjust density infill to a measurement range per patient specific parameters, and use 3D printing to form the implantable device based on said anatomical mode.

43. The system of claim 42, wherein the measurement range for the density infill is from 5 to 100%.
